(19) United States
(12) Patent Application Publication
(10) Pub. No.: US 2012/0020974 A1
(43) Pub. Date: Jan. 26, 2012

(54) OPTIMIZING ERYTHROPOIETIN THERAPY

(75) Inventors: Ravi Thadhani, Boston, MA (US); S. Ananth Karumanchi, Chestnut Hill, MA (US); Eliyahu Khankin, Boston, MA (US); Walter P. Mutter, West Newton, MA (US)

(73) Assignees: BETH ISRAEL DEACONESS MEDICAL CENTER, INC., Boston, MA (US); THE GENERAL HOSPITAL CORPORATION, Boston, MA (US)

(21) Appl. No.: 13/123,841

(22) PCT Filed: Oct. 16, 2009
(86) PCT No.: PCT/US09/61056

§ 371 Date: Oct. 5, 2011

Related U.S. Application Data
(60) Provisional application No. 61/106,444, filed on Oct. 17, 2008.

(51) Int. Cl. A61K 39/395; A61P 3/00; A61K 38/18; G01N 33/566; C12Q 3/00

(52) U.S. Cl. 424/139.1; 435/7.92; 436/501; 435/7.1; 435/3; 514/7.7

(57) ABSTRACT

The methods described herein can be used to identify ex ante which hemodialysis patients are likely to develop EPO resistance and therefore will require additional rHu EPO, and to provide treatments that can reduce the need for additional rHu EPO. In addition, the methods can be used to predict which subjects have a higher risk of mortality, to identify high-risk patients who can then be monitored more closely or treated more aggressively. Also provided are kits for carrying out the described methods.

### CLAIM OF PRIORITY

This application claims the benefit of U.S. Provisional Patent Application Ser. No. 61/106,444, filed on Oct. 17, 2008, the entire contents of which are hereby incorporated by reference.

### TECHNICAL FIELD

This invention relates to methods of optimizing erythropoietin therapy of subjects undergoing hemodialysis.

### BACKGROUND

Erythropoietin (Epo) is a protein hormone produced by the kidney in response to hypoxia. Epo binds the membrane bound erythropoietin receptor (EpoR) located on erythroblasts in the bone marrow. Following receptor binding an intracellular signaling cascade leads to the transcription of anti-apoptotic genes. This results in production of new red blood cells and improvement in anemia. Patients with end stage renal disease on dialysis have decreased renal Epo production and suffer from anemia. Recently, recombinant human erythropoietin (rHu EPO) has proved very useful in correcting the anemia of renal disease and improving quality of life outcomes in dialysis patients.

Two forms of rHu EPO are presently in common use, epoeitin alfa (EPOGEN, PROCRIT) and darbepoetin alfa (ARANESP) (see Nurko, Cleve. Clin. J. Med. 73(3):289-97 (2006)). Some patients respond well to Epo, but a subset of patients are resistant to Epo even at higher doses. This lack of response, termed “Erythropoietin Resistance,” is believed to be multifactorial and may be related to iron stores, bone disease, inflammation and other factors. Importantly, the requirement for higher doses of Epo has been associated with increased mortality. There is currently no way to accurately predict which patients will be resistant to Epo. In addition, treatment with rHu EPO is expensive, therefore optimizing the efficiency of its administration would be desirable.

### SUMMARY

The present invention is based, at least in part, on the discovery that circulating levels of soluble erythropoietin receptor levels (sEpoR) predict the amount of erythropoietin a hemodialysis subject will need to approach a target hematocrit.

Therefore, in one aspect, the invention provides methods that include determining sEpoR levels in a subject in need thereof, e.g., a subject who is undergoing or is about to undergo hemodialysis or who has anemia of chronic disease, e.g., in a sample from the subject, e.g., in a sample comprising serum from the subject. The determined sEpoR levels are compared to a reference, e.g., a threshold, above which the subject has a higher likelihood of needing additional doses of EPO to reach a therapeutic goal, i.e., a target hematocrit. The methods can also be used to assign subjects to a risk category, e.g., high risk or low risk, and to select subjects for a clinical trial, e.g., on the basis of high or low levels of sEpoR.

In an additional aspect, the invention provides methods of optimizing treatment for a subject who is undergoing or is about to undergo hemodialysis. The methods include determining one or more levels of sEpoR in the subject, e.g., at least one level of sEpoR at the start of hemodialysis. If the level is above a threshold, the methods include one or more of administering additional EPO to the subject; performing additional monitoring of the subject, e.g., of the subject's hematocrit; or assigning the subject to a risk category.

In yet another aspect, the invention provides methods for optimizing treatment of a subject who is undergoing or is about to undergo hemodialysis. The methods include determining one or more levels of sEpoR in the subject, e.g., at least one level of sEpoR at the start of hemodialysis. If the level is above a threshold, the methods include administering a treatment to reduce the levels of sEpoR in the blood, e.g., using an extracorporeal device such as an immunoabsorption device.

In a further aspect, the invention features methods of diagnosing, or determining a subject's risk of developing, resistance to erythropoietin (EPO). The methods include selecting a subject who is being or will be treated with EPO, e.g., a subject who is undergoing or about to undergo hemodialysis; obtaining a sample comprising serum from the subject; and determining a level of soluble EPO receptor (sEpoR) protein in the sample. The level of sEpoR in the sample is indicative of the subject's having or risk of developing EPO resistance. In some embodiments, the methods include comparing the level of sEpoR in the sample to a reference, e.g., a preselected threshold. In some embodiments, the the presence of a level of sEpoR in the sample that is above the reference indicates that the subject has, or has an increased risk of developing, resistance EPO.

In some embodiments, the methods further include one or more of the following:
assigning an identifier to the subject that correlates with the subject's risk; and/or
communicating information regarding the subject's risk to the subject's health insurance provider.

In some embodiments, if the level of sEpoR indicates that the subject has, or has an increased risk of developing, resistance to EPO, the methods further include one or more of the following:
administering an increased dose of EPO to the subject;
selecting the subject for increased monitoring, e.g., monitoring hemoglobin levels on a daily or weekly basis; and/or
administering a treatment to the subject to lower serum levels of sEpoR.

In another aspect, the invention also provides methods for increasing a subject's sensitivity to EPO, by administering a treatment to the subject that reduces levels of sEpoR in the serum of the subject. For example, the treatment can include removing blood from the subject; optionally separating serum or plasma from the blood; contacting the blood, plasma, or serum with an agent that selectively removes sEpoR from the blood; and returning the blood, plasma, or serum to the subject.

In some embodiments, the agent that selectively removes sEpoR from the blood is EPO or an anti-EPO antibody. The EPO or anti-EPO antibody can be immobilized on a substrate, e.g., a membrane or a bead.

Also provided by the present invention are devices including a housing forming a chamber having an inlet and an outlet configured for the passage of fluid through said chamber; a substrate, e.g., a membrane or a bead, disposed within the chamber; and immobilized on said substrate, an agent that selectively binds the EPO receptor, e.g., EPO or an EPO-receptor binding fragment thereof, or an antibody or antigen-binding portion thereof that selectively binds an EPO receptor, e.g., the soluble EPO receptor.

In yet another aspect, the invention features kits including an antibody or antigen-binding portion thereof that selectively binds to the soluble EPO receptor, and instructions for carrying out a method described herein.

In a further aspect, the invention provides isolated recombinant soluble EPO receptor polypeptides comprising an amino acid sequence at least 95% identical to SEQ ID NO:1, e.g., comprising SEQ ID NO:1, isolated nucleic acids encoding the polypeptides, vectors comprising the isolated nucleic acids, and host cells expressing the isolated nucleic acids. In some embodiments the isolated recombinant soluble EPO receptor polypeptides comprises an amino acid sequence at least 95% identical to amino acids 25-267 of SEQ ID NO:1, e.g., comprise amino acids 25-267 of SEQ ID NO:1.

In an additional aspect, the invention features isolated recombinant peptide comprising an amino acid sequence at least 95% identical to SEQ ID NO:2, e.g., comprising SEQ ID NO:2, isolated nucleic acids encoding the peptides, vectors comprising the isolated nucleic acids, and host cells expressing the isolated nucleic acids. In some embodiments, the isolated recombinant peptides further comprise an antigenic moiety, e.g., keyhole limpet haemocyanin (KLH) or bovine serum albumin (BSA).

Also provided herein are antibodies or antigen-binding fragments thereof that bind specifically to a soluble EPO receptor polypeptide as described herein, e.g., a polypeptide comprising SEQ ID NO:1, but do not bind to full length EPO receptor, and methods for generating those antibodies or antigen-binding fragments there, e.g., by immunizing an animal with a recombinant peptide comprising an amino acid sequence at least 95% identical to SEQ ID NO:2, e.g., comprising SEQ ID NO:2, optionally further comprising an additional antigenic moiety.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a bar graph showing the distribution of plasma sEpoR levels in the set of Incidence Chronic Hemodialysis patients (n=769).

FIG. 2 is a line graph showing hemoglobin levels at baseline, 90 days, and at 180 days according to sEpoR_high (>=1000 pg/ml, filled circles) and sEpoR_low (<1000 pg/ml) status (P=0.04, stars)

FIG. 3 is a line graph showing dose of EPO administered per hemodialysis (HD) session to sEpoR_high (>1000 pg/ml, circles) vs. sEpoR_low (<1000 pg/ml, stars) subjects (P=0.02 by repeated measures analysis).

FIG. 4 is a line graph showing total EPO administered to sEpoR_high (>1000 pg/ml, circles) vs. sEpoR_low (<1000 pg/ml, stars) subjects (P=0.04 by repeated measures analysis).

FIG. 5 is a line graph showing changes in weekly dose of EPO administered to sEpoR_high (>1000 pg/ml) vs. sEpoR_low (<1000 pg/ml) subjects (P=0.02 by repeated measures analysis).

FIG. 6 is a Western blot of BaF3 cell lysate showing increased phospho-Stat (p-Stat, top blot) with increased Epo concentration in mU/ml, while total Stat concentrations remained unchanged (Stat, bottom blot).

FIG. 7 is a Western blot of total BaF3/EpoR cell lysate showing blocking of phosphorylation of Stat5 while adding various doses of hr-sEpoR and activating cells with 5000 mU/mL of hrEpo.

FIG. 8 is a bar graph showing densitometry-estimated phosphorylated Stat5-to-total Stat5 ratios in Baf3/EpoR cells exposed to activation by 5000 mU/ml hrEpo in presence of various concentrations of hr-sEpoR.

FIG. 9 is a bar graph showing densitometry-estimated phosphorylated Stat5-to-total Stat5 ratios in Baf3/EpoR cells exposed to activation by 25 mU/ml hrEpo in presence of 10% patient serum. sEpoR-Low—patient with low levels of sEpoR; sEpoR-High1 and sEpoR-High2—patients with high levels of sEpoR.

FIG. 10A is a bar graph showing that IL-6, TNF-α and PMA increase sEpoR in the supernatant of K562 cells. K562 were plated in serum free media and exposed for 48 h to vehicle, PMA, IL-6 and TNF-α. At the end of the incubation cells were pelleted and the supernatant subjected to ELISA for sEpoR. sEpoR measurements were corrected for total protein concentration. * represents p value of <0.05 when compared to the control group.

FIG. 10B is a bar graph showing mean IL-6 levels in subjects with low (n=32) and high sEpoR (n=32) are shown. * represents p value of <0.05 when compared to low sEpoR group.

FIG. 11 is a schematic illustration of an immunoabsorptive device as described herein.

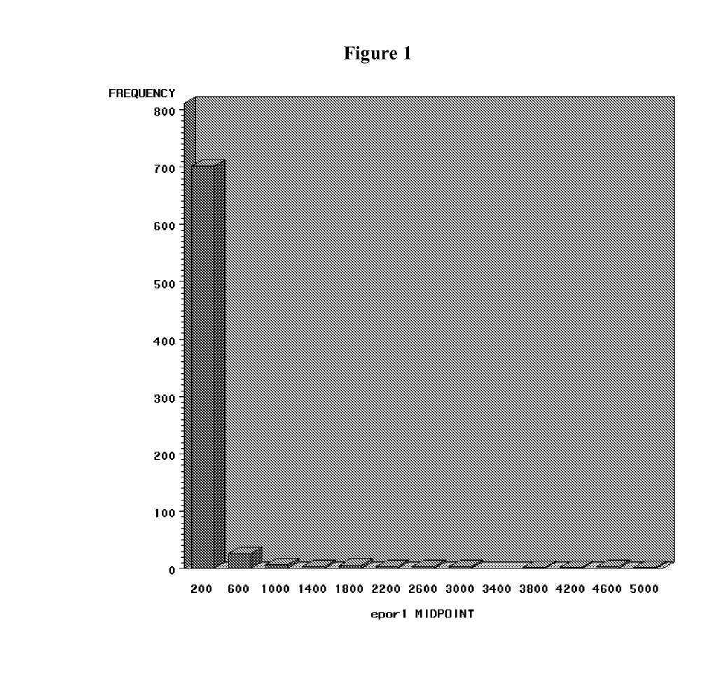

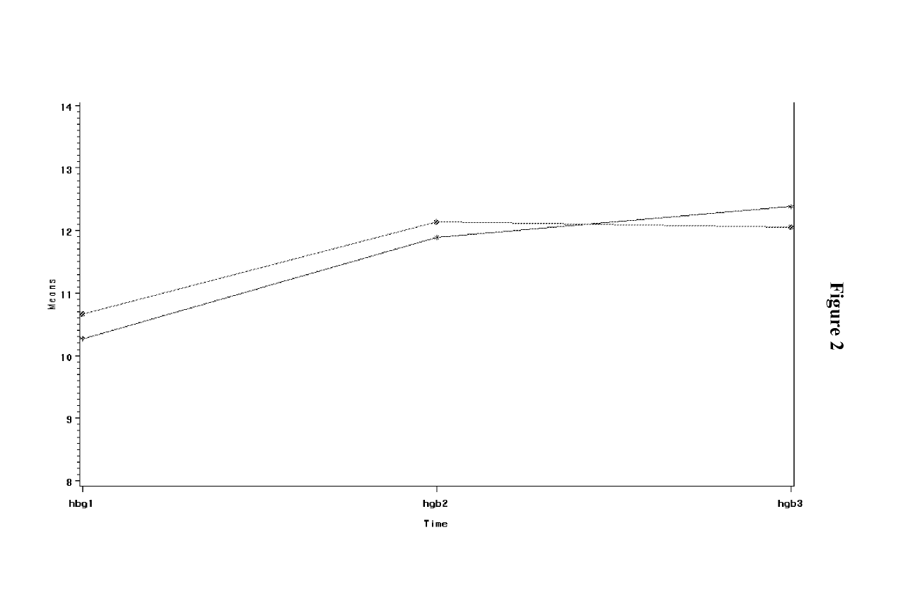

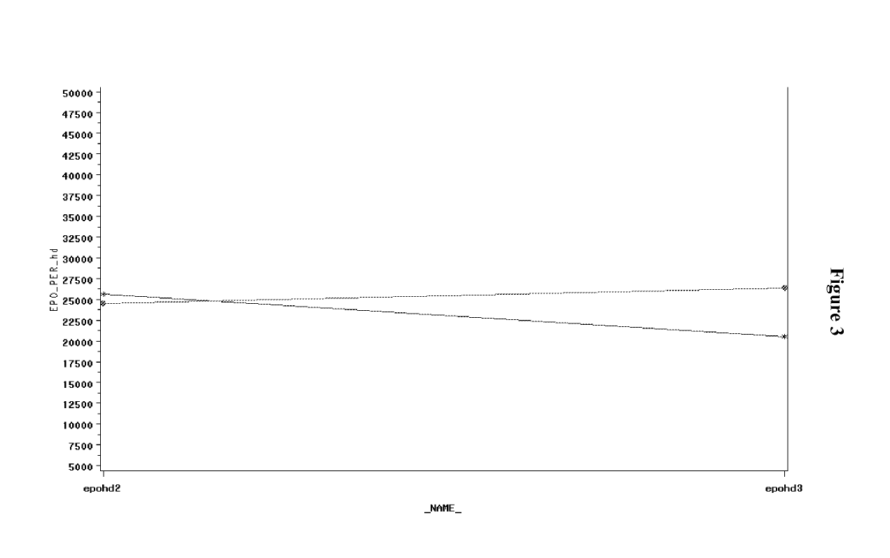

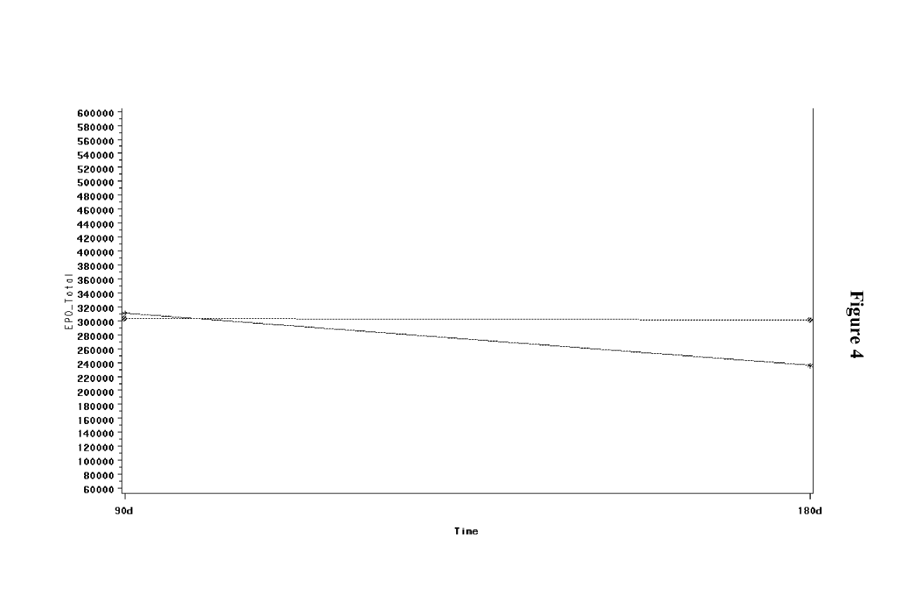

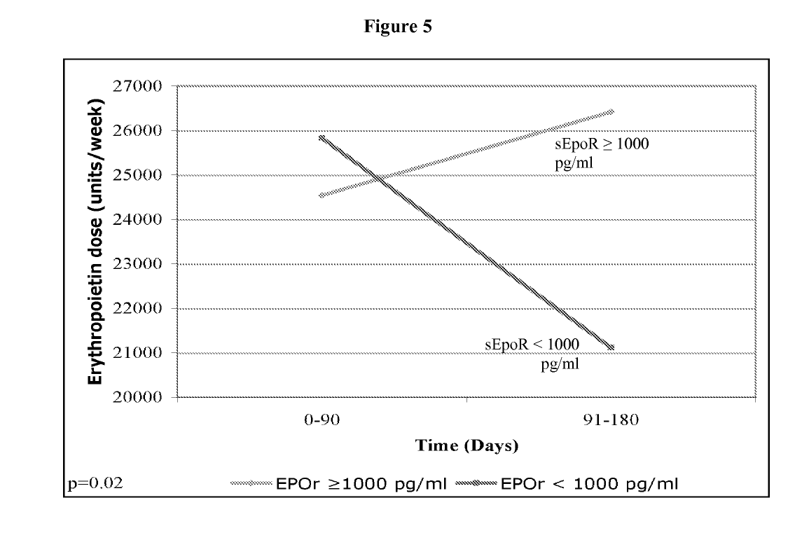

### DETAILED DESCRIPTION

The erythropoietin receptor (EpoR) is a membrane-bound receptor present in erythroblasts. Erythropoietin (Epo) binding causes phosphorylation of intracellular messengers leading to transcription of anti-apoptotic factors. Alternative mRNA splicing results in a soluble form of EpoR (sEpoR) present in human serum. The function of sEpoR is unknown, but levels are thought to correlate with the magnitude of erythropoiesis and sEpoR can block Epo signaling in vitro. No studies have systematically studied sEpoR levels in hemodialysis patients. As demonstrated herein, higher sEpoR levels at initiation of dialysis predict increased Epo dose requirements to achieve target hemoglobin levels.

The vast majority (some 60-80%) of hemodialysis patients will develop anemia during treatment that requires administration of an erythropoiesis-stimulating agent such as a recombinant human erythropoietin or analog thereof; these agents are referred to generally herein as rHu EPO and include epoeitin alfa (EPOGEN, PROCRIT) and darbepoetin alfa (ARANESP). Some patients who undergo such treatment develop resistance to the rHu EPO and require larger and larger doses to achieve the same (or diminished) therapeutic effect, e.g., a target hemoglobin level of about 10 g/dL or higher, e.g., 11-12 or 11-13 g/dl. The methods described herein can be used to identify ex ante which patients are likely to require additional rHu EPO, and to provide treatments that can reduce the need for additional rHu EPO.

The present methods allow for more strategic dosing protocols to achieve maximal effects with the minimal dose of Epo. This is especially important given recently identified off-target effects of Epo, including possible stimulation of tumor growth in patients with some malignancies. Moreover, using sEpoR levels to predict risk of mortality in dialysis patients is a useful way to identify high risk patients.

The EPO Receptor

The erythropoietin receptor (EPOr) is a 66 kD membrane-bound receptor, a member of the cytokine superfamily of type 1 transmembrane proteins, and is present in erythroblasts. Binding of erythropoietin (EPO) to the EPOr causes phosphorylation of intracellular messengers leading to nuclear transcription of anti-apoptotic factors. In erythroblasts, this promotes cell survival and proliferation leading to enhanced erythropoiesis.

EpoR is synthesized both as a membrane bound protein and in a soluble from (sEpoR) by alternative mRNA splicing (Baynes et al., Blood 82(7):2088-1095 (1993); Harris and Winkelman, Am. J. Hematol. 52:8-13 (1996)). The mRNA sequence of the full-length EPOr is available in the GenBank database at accession no. NM_{—}000121.2; the protein sequence is NP_{—}000112.1.

sEpoR may exist in one or more isoforms, e.g., of about 27-28 kD. The sequence of one isoform was determined and is shown below, in Example 4. Additional isoforms are described in the art, e.g., in Anagnostou et al., Proc. Nati. Acad. Sci. USA, 91:3974-3978 (1994);Todokoro et al., Gene. 106:283-284 (1991); Nakamura et al., Science 257:1138-1141 (1992); Binnie et al., Protein Exp. Pur. 11:271-278 (1997); Kuramochi et al., J. Mol. Biol. 216:567-575 (1990); Baynes et al., Blood 18:19a (1995, abstract suppl. 1); Zhan et al., Prot. Engineering, 12(6):505-513 (1999); Ehrenman and St. John, Exp. Hematol. 19:973-977 (1991); or Neumann et al., J. Biochem. 313:391-399 (1996), all incorporated herein by reference.

Secreted sEpoR is able to bind Epo. It has been identified in human serum and plasma as well as several tissues including brain, liver, spleen, kidney, heart and bone marrow. Recent data suggests that sEpoR plays a regulatory role in the brain as part of the ventilator acclimatization to hypoxia by binding endogenous Epo locally. It is well known that soluble receptors often play an important role in cytokine signaling by stabilizing their ligand, changing concentrations of active ligand or by altering the interaction between endogenous cytokine and membrane bound ligand.

The physiological role of the sEpoR has yet to be conclusively defined, as the literature contains conflicting reports on its effect on erythropoiesis (see, e.g., Baynes et al., (1993), supra; Yoshida et al., Blood 88:3246-3247 (1996); Harris and Winkelman, (1996), supra; and Westphal et al., Clin. Exp. Med. 2:45-52 (2002)).

Isolated sEpoR Nucleic Acid Molecules

In one aspect, the invention provides isolated nucleic acid molecules that encode a human sEpoR polypeptide described herein, e.g., SEQ ID NO:1 or amino acids 25-267 of SEQ ID NO:1 (amino acids 1-24 being a putative signal sequence). Also included are nucleic acid fragments encoding SEQ ID NO:2, fragments suitable for use as a hybridization probe, which can be used, e.g., to identify a nucleic acid molecule encoding a polypeptide of the invention, sEpoR mRNA, and fragments suitable for use as primers, e.g., PCR primers for the amplification or mutation of sEpoR nucleic acid molecules.

In one embodiment, an isolated nucleic acid molecule of the invention includes the nucleotide sequence shown in SEQ ID NO:3. In one embodiment, the nucleic acid molecule includes sequences encoding the human sEpoR protein (i.e., “the coding region”), as well as 5′ untranslated sequences.

In another embodiment, a sEpoR nucleic acid molecule includes a nucleic acid molecule which is a complement of a sequence described herein. In other embodiments, a sEpoR nucleic acid molecule is sufficiently complementary to a nucleotide sequence described herein that it can hybridize to the nucleotide sequence under stringent conditions, thereby forming a stable duplex.

In one embodiment, a isolated sEpoR nucleic acid molecule includes a nucleotide sequence which is at least about 85% or more homologous to the entire length of a nucleotide sequence shown in SEQ ID NO:3. In some embodiments, the nucleotide sequence is at least about 90%, 91%, 92%, 93%, 94%, 95%, 96%, 97%, 98%, 99% or 100% to SEQ ID NO:3.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Isolated sEpoR Polypeptides

In another aspect, the invention features isolated recombinant sEpoR polypeptides, or fragments thereof, e.g., unique portions useful as immunogens or antigens to raise or test (or more generally to bind) anti-sEpoR specific antibodies. Recombinant sEpoR protein can be isolated from cells or tissue sources using standard protein purification techniques. sEpoR protein or fragments thereof can be produced by recombinant DNA techniques or synthesized chemically.

The polypeptides can be expressed in systems, e.g., cultured cells, which result in substantially the same post-translational modifications present when expressed the polypeptide is expressed in a native (human) cell, or in systems which result in the alteration or omission of post-translational modifications, e.g., glycosylation or cleavage, present when expressed in a native cell.

In some embodiments the sEpoR protein, or fragment thereof comprises amino acids 25-267 of SEQ ID NO:1, or SEQ ID NO:1, or differs from the corresponding sequence in amino acids 25-267 of SEQ ID NO:1, or in SEQ ID NO:1, by at least one but by less than 15, 10 or 5 amino acid residues, or less than 20%, 15%, 10% or 5% of the residues in it differ from the corresponding sequence in amino acids 25-267 of SEQ ID NO:1, or in SEQ ID NO:1. (If this comparison requires alignment the sequences should be aligned for maximum homology. “Looped” out sequences from deletions or insertions, or mismatches, are considered differences.) The differences are, preferably, differences or changes at a non essential residue or a conservative substitution. In a preferred embodiment the differences are not in the EPO receptor ligand binding domain, e.g., about amino acids 27-140 of SEQ ID NO:1, and/or in the Fibronectin type 3 domain, e.g., about amino acids 146-244, and/or in the sEpoR specific domain, which is SEQ ID NO:2.

Other embodiments include a protein that contain one or more changes in amino acid sequence, e.g., a change in an amino acid residue which is not essential for activity. Such sEpoR proteins differ in amino acid sequence from SEQ ID NO:1, yet retain biological activity. In some embodiments, the protein includes an amino acid sequence at least about 80%, 85%, 90%, 95%, 98% or more homologous to amino acids 25-267 of SEQ ID NO:1, e.g., to SEQ ID NO:1.

A sEpoR protein or fragment is provided which varies from the sequence of SEQ ID NO:1 by at least one but by less than 15, 10 or 5 amino acid residues in the protein or fragment but which does not differ from SEQ ID NO:1 in the EPO receptor ligand binding domain, e.g., about amino acids 27-140 of SEQ ID NO:1, and/or in the Fibronectin type 3 domain, e.g., about amino acids 146-244, and/or in the sEpoR specific domain, which is SEQ ID NO:2. (If this comparison requires alignment the sequences should be aligned for maximum homology. “Looped” out sequences from deletions or insertions, or mismatches, are considered differences.) In some embodiments the difference is at a non essential residue or is a conservative substitution, while in others the difference is at an essential residue or is a non conservative substitution.

In a preferred embodiment, the sEpoR protein has an amino acid sequence shown in SEQ ID NO:1. In other embodiments, the sEpoR protein is substantially identical to SEQ ID NO:1. In yet another embodiment, the sEpoR protein is substantially identical to SEQ ID NO:1 and retains the functional activity of the protein of SEQ ID NO:1, e.g., the ability to bind EPO. Methods for making soluble proteins are known in the art, see, e.g., Binnie et al., (1997) supra, or Zhan et al., (1999) supra, inter alia.

sEpoR Chimeric or Fusion Proteins

In another aspect, the invention provides sEpoR chimeric or fusion proteins. As used herein, a sEpoR “chimeric protein” or “fusion protein” includes a sEpoR polypeptide or fragment thereof (e.g., a fragment comprising all or part of SEQ ID NO:2) linked to a non-sEpoR polypeptide. A “non-sEpoR polypeptide” refers to a polypeptide having an amino acid sequence corresponding to a protein which is not substantially homologous to the sEpoR protein, e.g., a protein which is different from the sEpoR protein and which is derived from the same or a different organism. The sEpoR polypeptide of the fusion protein can correspond to all or a portion e.g., a fragment described herein of a sEpoR amino acid sequence. In a preferred embodiment, a sEpoR fusion protein includes at least one (or two) biologically active portion of a sEpoR protein (e.g., SEQ ID NO:2 or an antigenic fragment thereof, e.g., at least 10, 11, 12, 13, 14, 15, 16, 17, 18, 19, 20, 21, 22, or more consecutive amino acids of SEQ ID NO:2). The non-sEpoR polypeptide can be fused to the N-terminus or C-terminus of the sEpoR polypeptide or fragment thereof.

The fusion protein can include a moiety which has a high affinity for a ligand. For example, the fusion protein can be a GST-sEpoR fusion protein in which the sEpoR sequences are fused to the C-terminus of the GST sequences. Such fusion proteins can facilitate the purification of recombinant sEpoR. Alternatively, the fusion protein can be a sEpoR protein containing a heterologous signal sequence at its N-terminus. In certain host cells (e.g., mammalian host cells), expression and/or secretion of sEpoR can be increased through use of a heterologous signal sequence.

Fusion proteins can include all or a part of a serum protein, e.g., an IgG constant region, or human serum albumin.

The sEpoR fusion proteins of the invention can be incorporated into pharmaceutical compositions and administered to a subject in vivo. The sEpoR fusion proteins can be used to affect the bioavailability of a sEpoR substrate. sEpoR fusion proteins may be useful therapeutically for the treatment of disorders caused by, for example, (i) aberrant modification or mutation of a gene encoding a sEpoR protein; (ii) mis-regulation of the sEpoR gene; and (iii) aberrant post-translational modification of a sEpoR protein.

Moreover, the sEpoR-fusion proteins of the invention can be used as immunogens to produce anti-sEpoR antibodies in a subject, to purify sEpoR ligands and in screening assays to identify molecules which inhibit the interaction of sEpoR with a sEpoR substrate.

Expression vectors are commercially available that already encode a fusion moiety (e.g., a GST polypeptide). A sEpoR-encoding nucleic acid can be cloned into such an expression vector such that the fusion moiety is linked in-frame to the sEpoR protein.

Recombinant Expression Vectors, Host Cells and Genetically Engineered Cells

In another aspect, the invention includes, vectors, preferably expression vectors, containing a nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a sEpoR nucleic acid as described herein in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., sEpoR proteins, mutant forms of sEpoR proteins, fusion proteins, and the like).

The recombinant expression vectors of the invention can be designed for expression of sEpoR proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRITS (Pharmacia, Piscataway, N.J.) which fuse glutathione S-transferase (GST), maltose E binding protein, or protein A, respectively, to the target recombinant protein.

Purified fusion proteins can be used in sEpoR activity assays, (e.g., direct assays or competitive assays described in detail below), or to generate antibodies specific for sEpoR proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The sEpoR expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the—fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., (1986) Antisense RNA as a molecular tool for genetic analysis, Reviews—Trends in Genetics 1:1.

Another aspect the invention provides a host cell which includes a nucleic acid molecule described herein, e.g., a sEpoR nucleic acid molecule within a recombinant expression vector or a sEpoR nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a sEpoR protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a sEpoR protein. Accordingly, the invention further provides methods for producing a sEpoR protein using the host cells of the invention. In one embodiment, the method includes culturing the host cell of the invention (into which a recombinant expression vector encoding a sEpoR protein has been introduced) in a suitable medium such that a sEpoR protein is produced. In another embodiment, the method further includes isolating a sEpoR protein from the medium or the host cell.

In another aspect, the invention features, a cell or purified preparation of cells which include a sEpoR transgene, or which otherwise misexpress sEpoR. The cell preparation can consist of human or non human cells, e.g., rodent cells, e.g., mouse or rat cells, rabbit cells, or pig cells. In preferred embodiments, the cell or cells include a sEpoR transgene, e.g., a heterologous form of a sEpoR, e.g., a gene derived from humans (in the case of a non-human cell). The sEpoR transgene can be misexpressed, e.g., overexpressed or underexpressed. In other preferred embodiments, the cell or cells include a gene which misexpress an endogenous sEpoR, e.g., a gene the expression of which is disrupted, e.g., a knockout. Such cells can serve as a model for studying disorders which are related to mutated or mis-expressed sEpoR alleles or for use in drug screening.

In another aspect, the invention features, a human cell, e.g., a hematopoietic stem cell, transformed with nucleic acid which encodes a subject sEpoR polypeptide.

Also provided are cells, preferably human cells, e.g., human hematopoietic or fibroblast cells, in which an endogenous sEpoR is under the control of a regulatory sequence that does not normally control the expression of the endogenous sEpoR gene. The expression characteristics of an endogenous gene within a cell, e.g., a cell line or microorganism, can be modified by inserting a heterologous DNA regulatory element into the genome of the cell such that the inserted regulatory element is operably linked to the endogenous sEpoR gene. For example, an endogenous sEpoR gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

Methods of Diagnosing, or Predicting Development of, Resistance to rHu EPO

As described herein, circulating levels of sEpoR can be used to diagnose resistance, or predict which subjects are most likely to develop resistance, to rHu EPO, and therefore which subjects need or are most likely to need an increased dosing regimen. For example, a level of sEpoR can be determined before or after the subject starts a hemodialysis regimen, and the presence of resistance or risk of developing resistance can be calculated based on the level (e.g., in comparison to a threshold level). In some embodiments, the level of sEpoR can be determined more than once, e.g., before, during, and after dialysis or other treatments.

In some embodiments, the methods described herein include determining levels of other EpoR fragments, e.g., proteolytic cleavage fragments, in addition to or instead of sEpoR.

In some embodiments, the methods can include using the level of sEpoR determined as described herein in combination with a level of another biomarker, e.g., in a ratio. For example, the methods can include using a ratio of sEpoR/hemoglobin or sEpoR/weekly EPO dose, and optionally optimizing the subject's EPO dose, or administering another treatment described herein, if the ratio indicates the presence or increased risk of developing resistance to EPO.

Methods of Measuring sEpoR

The predictive methods described herein generally include a step of diagnosing or predicting or determining a subject's risk of developing EPO resistance based on a level of sEpoR in the subject. A number of methods are known in the art for determining the level of sEpoR. In general, sEpoR levels will be determined in a sample of serum or plasma from the subject, but whole blood can also be used; in this case, it will generally be desirable to use a method that specifically detects the soluble form of sEpoR, to minimize any effect of full-length EPOr on the measurement. In some embodiments, an immunoassay is used, e.g., an enzyme linked immunosorbent assays (ELISAs), immunoprecipitations, immunofluorescence, enzyme immunoassay (EIA), radioimmunoassay (RIA), or Western blot analysis. In a preferred embodiment, an ELISA is used.

The methods can also include a purification step, for example a step that removes non-protein components from the sample, and/or that removes proteins of the wrong size from the sample, before the assay is performed. For example, a step that would remove full length EpoR can be performed, e.g., using size fractionation.

Additional Biomarkers

The present methods can also include evaluating levels of other biomarkers in serum, as an alternative or in addition to sEpoR. For example, levels of IL-6 and/or TNF-alpha can be measured. Elevated levels of either IL-6 or TNF-alpha indicate the presence of an increased likelihood of elevated levels of sEpoR, and thus of an increased risk of EPO resistance (and an increased need for EPO administration). Methods for measuring IL-6 and TNF-alpha levels in serum are known in the art, and can include the same general methods that can be used for measuring sEpoR, e.g., as described above.

sEpoR-Specific Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments, which retain the ability to bind antigen. Such fragments can be obtained commercially, or using methods known in the art. For example F(ab)2 fragments can be generated by treating the antibody with an enzyme such as pepsin, a non-specific endopeptidase that normally produces one F(ab)2 fragment and numerous small peptides of the Fc portion. The resulting F(ab)2 fragment is composed of two disulfide-connected Fab units. The Fc fragment is extensively degraded and can be separated from the F(ab)2 by dialysis, gel filtration or ion exchange chromatography. F(ab) fragments can be generated using papain, a non-specific thiol-endopeptidase that digests IgG molecules, in the presence of a reducing agent, into three fragments of similar size: two Fab fragments and one Fc fragment. When Fc fragments are of interest, papain is the enzyme of choice because it yields a 50,00 Dalton Fc fragment; to isolate the F(ab) fragments, the Fc fragments can be removed, e.g., by affinity purification using protein A/G. A number of kits are available commercially for generating F(ab) fragments, including the ImmunoPure IgG1 Fab and F(ab′)₂ Preparation Kit (Pierce Biotechnology, Rockford, Ill.). In addition, commercially available services for generating antigen-binding fragments can be used, e.g., Bio Express, West Lebanon, N.H.

The antibody can be a polyclonal, monoclonal, recombinant, e.g., a chimeric, de-immunized or humanized, fully human, non-human, e.g., murine, or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. The antibody can be coupled to a toxin or imaging agent.

General methods for making antibodies are known in the art. To make sEpoR-specific antibodies, an antigenic peptide comprising an N-terminal sequence of sEpoR that is not present in the full length EpoR can be used as an immunogen, e.g., a peptide comprising the sequence GEAPGGGVGGARANHGASPPP (SEQ ID NO:2), or at least 80%, e.g,. 90 or 95% of the full length of SEQ ID NO:2. In some embodiments, an sEpoR-specific antibody binds specifically to SEQ ID NO:1 or SEQ ID NO:2, but does not bind to a full-length EpoR.

Methods for making monoclonal antibodies are known in the art. Basically, the process involves obtaining antibody-secreting immune cells (lymphocytes) from the spleen of a mammal (e.g., mouse) that has been previously immunized with the antigen of interest (e.g., a cancer-related antigen) either in vivo or in vitro. The antibody-secreting lymphocytes are then fused with myeloma cells or transformed cells that are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Mammalian lymphocytes are immunized by in vivo immunization of the animal (e.g., a mouse) with a cancer-related antigen. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by known techniques, for example, using polyethylene glycol (“PEG”) or other fusing agents (See Milstein and Kohler, Eur. J. Immunol. 6:511 (1976), which is hereby incorporated by reference). This immortal cell line, which is preferably murine, but can also be derived from cells of other mammalian species, including but not limited to rats and humans, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also known. Typically, such antibodies can be raised by administering the protein or polypeptide of the present invention subcutaneously to New Zealand white rabbits that have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized, e.g., with pentobarbital 150 mg/Kg IV. This and other procedures for raising polyclonal antibodies are disclosed in E. Harlow, et. al., editors, Antibodies: A Laboratory Manual (1988).

In addition to utilizing whole antibodies, the invention encompasses the use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Acad. Press 1983).

Chimeric, humanized, de-immunized, or completely human antibodies are desirable for applications which include repeated administration, e.g., therapeutic treatment of human subjects.

Chimeric antibodies generally contain portions of two different antibodies, typically of two different species. Generally, such antibodies contain human constant regions and variable regions from another species, e.g., murine variable regions. For example, mouse/human chimeric antibodies have been reported which exhibit binding characteristics of the parental mouse antibody, and effector functions associated with the human constant region. See, e.g., U.S. Pat. Nos. 4,816,567; 4,978,745; 4,975,369; and 4,816,397, all of which are incorporated by reference herein. Generally, these chimeric antibodies are constructed by preparing a genomic gene library from DNA extracted from pre-existing murine hybridomas (Nishimura et al., Cancer Research, 47:999 (1987)). The library is then screened for variable region genes from both heavy and light chains exhibiting the correct antibody fragment rearrangement patterns. Alternatively, cDNA libraries are prepared from RNA extracted from the hybridomas and screened, or the variable regions are obtained by polymerase chain reaction. The cloned variable region genes are then ligated into an expression vector containing cloned cassettes of the appropriate heavy or light chain human constant region gene. The chimeric genes can then be expressed in a cell line of choice, e.g., a murine myeloma line. Such chimeric antibodies have been used in human therapy.

Humanized antibodies are known in the art. Typically, “humanization” results in an antibody that is less immunogenic, with complete retention of the antigen-binding properties of the original molecule. In order to retain all the antigen-binding properties of the original antibody, the structure of its combining-site has to be faithfully reproduced in the “humanized” version. This can potentially be achieved by transplanting the combining site of the nonhuman antibody onto a human framework, either (a) by grafting the entire nonhuman variable domains onto human constant regions to generate a chimeric antibody (Morrison et al., Proc. Natl. Acad. Sci., USA 81:6801 (1984); Morrison and Oi, Adv. Immunol. 44:65 (1988) (which preserves the ligand-binding properties, but which also retains the immunogenicity of the nonhuman variable domains); (b) by grafting only the nonhuman CDRs onto human framework and constant regions with or without retention of critical framework residues (Jones et al. Nature, 321:522 (1986); Verhoeyen et al., Science 239:1539 (1988)); or (c) by transplanting the entire nonhuman variable domains (to preserve ligand-binding properties) but also “cloaking” them with a human-like surface through judicious replacement of exposed residues (to reduce antigenicity) (Padlan, Molec. Immunol. 28:489 (1991)).

Humanization by CDR grafting typically involves transplanting only the CDRs onto human fragment onto human framework and constant regions. Theoretically, this should substantially eliminate immunogenicity (except if allotypic or idiotypic differences exist). However, it has been reported that some framework residues of the original antibody also need to be preserved (Riechmann et al., Nature 332:323 (1988); Queen et al., Proc. Natl. Acad. Sci. USA 86:10,029 (1989)). The framework residues which need to be preserved can be identified by computer modeling. Alternatively, critical framework residues may potentially be identified by comparing known antibody combining site structures (Padlan, Molec. Immun. 31(3):169-217 (1994)). The invention also includes partially humanized antibodies, in which the 6 CDRs of the heavy and light chains and a limited number of structural amino acids of the murine monoclonal antibody are grafted by recombinant technology to the CDR-depleted human IgG scaffold (Jones et al., Nature 321:522-525 (1986)).

Deimmunized antibodies are made by replacing immunogenic epitopes in the murine variable domains with benign amino acid sequences, resulting in a deimmunized variable domain. The deimmunized variable domains are linked genetically to human IgG constant domains to yield a deimmunized antibody (Biovation, Aberdeen, Scotland).

The antibody can also be a single chain antibody. A single-chain antibody (scFV) can be engineered (see, for example, Colcher et al., Ann. N. Y. Acad. Sci. 880:263-80 (1999); and Reiter, Clin. Cancer Res. 2:245-52 (1996)). The single chain antibody can be dimerized or multimerized to generate multivalent antibodies having specificities for different epitopes of the same target protein. In some embodiments, the antibody is bivalent or monovalent, e.g., as described in Abbs et al., Ther. Immunol. 1(6):325-31 (1994), incorporated herein by reference.

In some embodiments, the anti-sEpoR antibodies are not neutralizing antibodies, do not bind to the full-length EpoR, and/or are engineered for rapid clearance from the blood stream.

Methods of Optimizing Treatment—Methods and Devices for Reducing Resistance to rHu EPO

In those subjects identified as having high circulating levels of sEpoR, one or more treatments can be administered. For example, in a subject with high levels of sEpoR, the method can include increasing or administering a high dose of EPO. Alternatively or in addition, the methods can include administering a treatment to reduce the levels of sEpoR. For example, in combination with their normal hemodialysis, e.g., as part of the dialysis circuit, one or more devices can be used, e.g., immunoabsorptive devices. An exemplary device is shown in FIG. 11. Immunoabsorptive device 100 includes a cartridge 101 including a 6chamber disposed within the cartridge (shown in cutaway 104), and a substrate (e.g., a membrane or beads 105) disposed within the chamber, and immobilized on the substrate is an agent that binds the sEpoR. Further, the chamber includes at least a fluid inlet 102 and a fluid outlet 103, which are configured to be connected to the dialysis circuit for fluid flow such that blood, serum, or plasma from the subject enters into the chamber via the inlet, contacts the agent immobilized on the substrate, and exits the chamber via the outlet (fluid flow path shown by arrows in FIG. 11). When it is desirable to have only plasma or serum from the subject contact the chamber, the dialysis circuit can further include a device for separation of the desired fluid from the whole blood of the subject, and a device for recombining the fluid with the blood after it passes through the chamber.

In one example, the agent that binds sEpoR can be an anti-EPO antibody, e.g., as described in Harris and Winkelmann, (1996) supra, Westphal et al., (2002) supra, or Baynes et al., (1993) supra. In a preferred embodiment, the agent that binds sEpoR is an antibody that binds specifically to sEpoR, e.g., to an N-terminal sequence of the sEpoR that is not present in the full length EpoR. As one example, the antibody may bind specifically to SEQ ID NO:1, but not to full length sEpoR, or may bind specifically to another isoform of sEpoR, e.g., as known in the art, e.g., as described in Anagnostou et al., Proc. Nati. Acad. Sci. USA, 91:3974-3978 (1994);Todokoro et al., Gene. 106:283-284 (1991); Nakamura et al., Science 257:1138-1141 (1992); Binnied et al., Protein Exp. Pur. 11:271-278 (1997); Kuramochi et al., J. Mol. Biol. 216:567-575 (1990); Baynes et al., Blood 18:19a (1995, abstract suppl. 1); Ehrenman and St. John, Exp. Hematol. 19:973-977 (1991); or Neumann et al., J. Biochem. 313:391-399 (1996), all of which are incorporated herein by reference, but not to full length EpoR. Alternatively, the agent that binds the sEpoR can be EPO itself, e.g., recombinant EPO.

In cases where an agent (e.g., anti-EPOr antibody or EPO) is used that binds to the full length EPOr as well as the sEpoR, the dialysis circuit can be configured such that serum or plasma is separated from EPOr-expressing blood cells before being contacted with the device, to avoid saturating the device with EPOr present on the surface of the cells (and undesirably removing those cells from the circulation).

Methods for making immunabsorptive devices are known in the art, see, e.g., Ameer et al., Kidney Int. 59(4):1544-1550 (2001); Sulowicz and Stompor, Neph. Dial. Transpl. 18(Supp. 5):v59 (2003); and U.S. Pat. No. 4,770,774. Such devices can be configured for use in any standard dialysis or apheresis device. See, e.g., U.S. Pat. Nos. 6,544,727; 5,750,025; and 5,637,082. In vivo therapeutic apheresis can be carried out using a filter device adapted for being implanted in a blood vessel, e.g., as described in U.S. Pat. No. 7,267,771. General methods known in the art for making bioartificial liver assist devices can also be used.

In addition, the methods can include reducing the level of inflammation in the subject, e.g., by administering an anti-inflammatory treatment, sufficient to reduce levels of IL-6 and/or TNF-alpha in the subject; the administration of such an anti-inflammatory treatment is expected to reduce sEpoR levels.

The effect of a treatment to reduce sEpoR levels as described herein can be monitored by measuring sEpoR levels multiple times, e.g., before and after such treatment is administered. In addition, the subject can be monitored by determining hematocrit and/or hemoglobin levels, e.g., before, during, and/or after such treatment is administered. In some embodiments, the treatment is continued or repeated until a circulating level below a preselected threshold is obtained.

Kits

Also provided herein are kits useful in carrying out the methods described herein. Such kits include an agent that selectively binds the binds to the soluble EPO receptor, i.e., does not significantly bind the full-length EpoR, such as an antibody or antigen-binding fragment thereof that recognizes the sEpoR. Useful antigen-binding portions are known in the art and include F(ab) and F(ab′)2 fragments which can be generated by treating the antibody with an enzyme such as pepsin. The antibodies can be polyclonal, or more preferably, monoclonal. The antibody or fragment thereof can be labeled, e.g., directly coupled (i.e., physically linked) to a detectable substance, or indirectly labeled by reactivity with a detectable substance. A number of suitable detectable substances are known in the art.

In general the kits will also include a standard that can be used to quantify levels of sEpoR in the sample, e.g., to determine whether the levels of sEpoR are above a selected threshold. The selected threshold may vary depending on the assay used. In general, a value greater than 2 standard deviations above the normal distribution of soluble Epo receptor is used to define the selected threshold. In some embodiments, the selected threshold is, e.g., about 1000, 900, 800, 700, or 600 pg/ml, and can vary depending on the a number of factors, e.g., the assay used, sample preparation methods, and population distributions. An appropriate threshold can be determined by one of skill in the art using routine methods. The compound or agent can be packaged in a suitable container. Optionally, the kit can also include an agent, e.g., an antibody or antigen-binding portion thereof, that binds the full-length EpoR. The kits further include instructions for carrying out a method described herein.

In some embodiments, the kits can include: (1) a first antibody (e.g., attached to a solid support) that binds selectively and specifically to sEpoR; and, optionally, (2) a second, different antibody which binds to either the polypeptide or the first antibody and is conjugated to a detectable agent. The kits can also includes a buffering agent, a preservative, or a protein stabilizing agent. The kits can also includes components necessary for detecting the detectable agent (e.g., an enzyme or a substrate). The kits can also contain a control sample or a series of control samples which can be assayed and compared to the test sample contained. Each component of the kits can be enclosed within an individual container, and the various containers can be within a single package, along with instructions for interpreting the results of assays performed using the kit.

### EXAMPLES

The invention is further described in the following examples, which do not limit the scope of the invention described in the claims.

### Example 1

### Circulating sEpoR Receptor in Humans

This study was performed to determine whether high soluble EPOr levels at initiation of dialysis predict increased EPO dosing requirements to achieve target hematocrit.

Accelerated Mortality on Renal Replacement (ArMORR) is a nationally representative prospective cohort study of incident hemodialysis patients (described in Wulf et al., Kidney Int. 72(8):1004-1013 (2007)). This cohort consisted of subjects throughout the US, and reflects the general dialysis population in the US. 766 patients who survived at least 180 days were selected at random from the ArMORR database (n=10,044). sEpoR level was measured at the start of dialysis (Duo Set ELISA, R&D Systems). The primary outcome was average EPO dose per week. The cohort was divided by baseline sEpoR levels; those with sEpoR levels of 1,000 pg/ml or higher were identified as having “high” levels (n=33, vs. n=733). (A significant difference in Epo dosing was also found using a threshold of 800 pg/ml.) Mixed linear models were used to compare average EPO dose per week by baseline sEpoR. Multivariate models were adjusted for body mass index and gender.

No difference in age, race, gender, BMI, blood pressure, albumin, parathyroid hormone, ferritin, transferrin saturation or hemoglobin was found between high and low sEpoR groups.

Mean weekly EPO dose was not different in the 0-90 day period (24,200±13,808 vs. 25,151±14,380) in the high and low sEpoR groups respectively. However, EPO dose was significantly increased in the high sEpoR group during the 91-180 day period (26,417±22,973 vs. 21,101±19,027, p=0.02). Hemoglobin was not statistically different.

These results demonstrate that high soluble EPOr levels at initiation of dialysis do predict increased EPO dose requirements to achieve target hematocrit. Circulating EPOr may directly inhibit EPO mediated signaling and thereby contribute to the increased EPO dose needed in dialysis patients with EPO resistance.

### Example 2

### sEpoR Blocks Epo Mediated Cell Signaling

This Example describes experiments done to determine whether human serum with high levels of sEpoR blocks Epo mediated cell signaling.

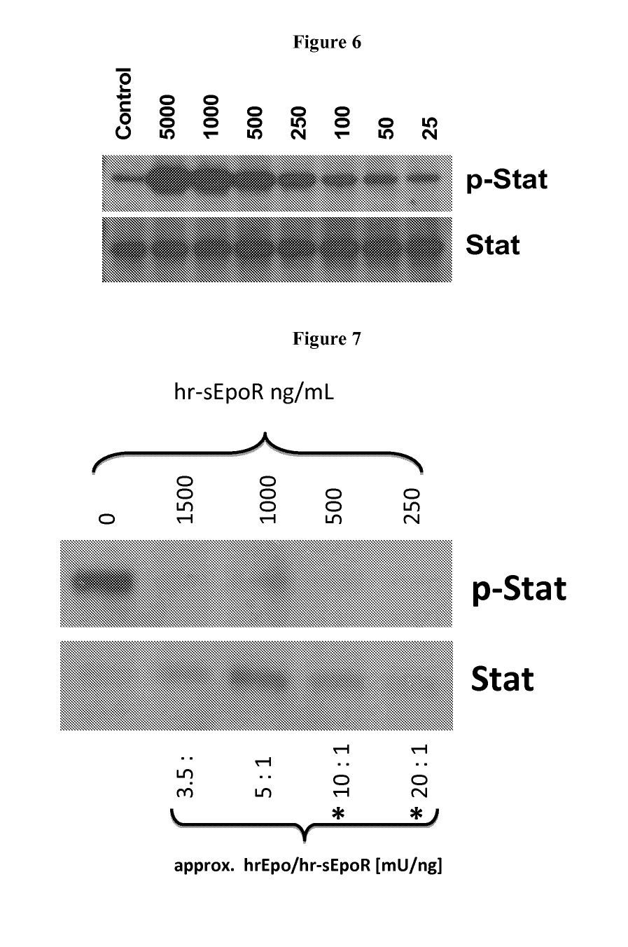

Proliferation assays with BaF3 murine pro B cells stably transfected with EpoR (BaF3/EpoR) were conducted to test the hypothesis that serum with high levels of sEpoR blocks Epo mediated proliferation. This cell line is well characterized and proliferates in response to Epo. Phosphorylation of Stat, a down stream signaling molecule, is used as a reporter of Epo-induced signaling. The cells show a dose-dependent increase in Stat phosphorylation in response to Epo (FIG. 6). As shown in FIG. 7, this phosphorylation of StatS was blocked by the addition of various doses of human recombinant sEpoR (hr-sEpoR) (obtained from SIGMA-Aldrich, St. Louis, Mo.) in the presence of an activating amount of rhEpo, 5000 mU/mL. The asterisks in FIG. 7 indicate those doses with hrEpo/hr-sEpoR ratios comparable to the in vivo findings described above, e.g., approx. 25 mU/mL of Epo and about 4 ng/mL of sEpoR.

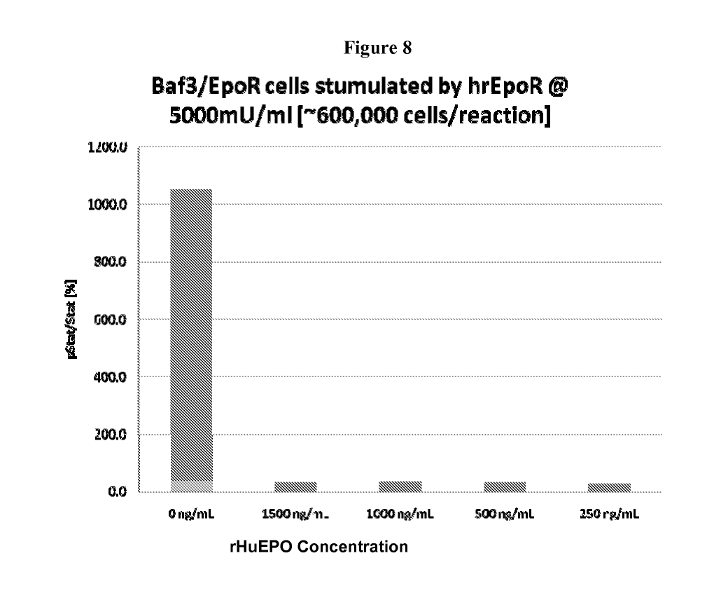

Densitometry was used to estimate ratios of phosphorylated Stat5-to-total Stat5 in Baf3/EpoR cells exposed to activation by 5000 mU/ml hrEpo (which, as shown in FIG. 6, produces a robust phosphorylation response) in the presence of various concentrations of hr-sEpoR. FIG. 8 shows that the presence of levels of hr-sEpoR above 1500 ng/ml significantly blocked the EPO-induced Stat phosphorylation.

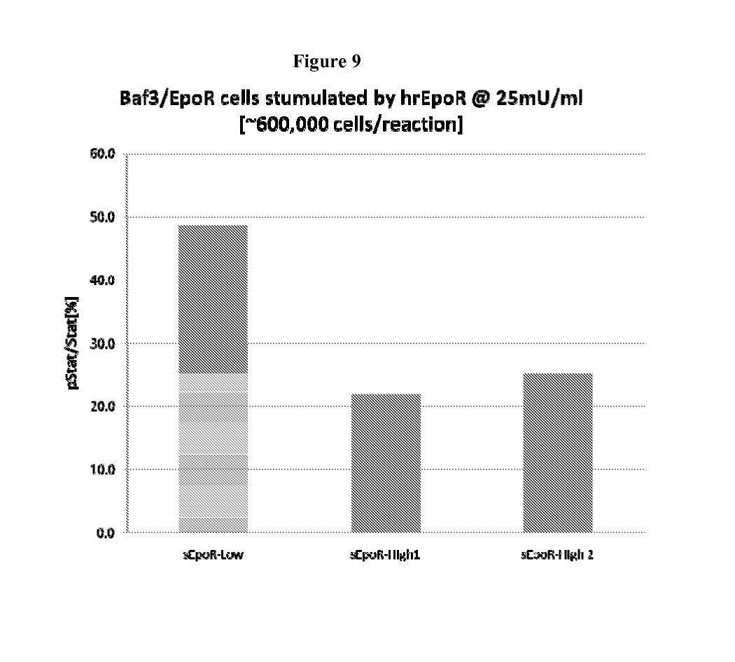

Finally, the effect of serum from human patients with known levels of hr-sEpoR was evaluated using the same system. As shown in FIG. 9, serum from hemodialysis patients with high levels of hr-sEpoR significantly reduced EPO-induced Stat phosphorylation when compared with serum from hemodialysis patients with relatively low levels of sEpoR, under conditions of activation with physiologic levels of hrEpo (25 mU/mL).

These results demonstrate that high levels of sEpoR block Epo-mediated cell signaling, as indicated by a reduction in downstream signalling. This supports the concept that sEpoR levels may identify those at risk of requiring higher Epo doses, those who may not respond to standard doses, and/or those who may benefit from interventions to reduce sEpoR levels in blood.

### Example 3

### sEpoR is Regulated by Proinflammatory Cytokines

The source of circulating sEpoR is unknown. It is plausible that sEpoR levels are regulated, and that sEpoR has physiologic function in some tissues. Some have noted a positive correlation between sEpoR levels and the degree of erythropoiesis (Baynes et al., Blood 82:2088-2095 (1993)) while others have not observed an association (Yoshida et al., Blood 88:3246-3247 (1996)). In addition, recent data shows a correlation between elevated TNF-α, IL-6 and IL-8 levels and anemia in patients with chronic kidney disease (Maruyama et al., J Gene Med 6, 228-237 (2004)). It was hypothesized that sEpoR may be stimulated by inflammatory cytokines present in the uremic patients.

To test this hypothesis, K562 cells, a cell line known to express EpoR (Nagao et al., Biochem Biophys Res Commun 188:888-897 (1992); Maiese et al., JAMA 293:90-95 (2005)), were selected. Phorbol ester (PMA) was used as a positive control as it has been shown to induce secretion of soluble growth factor receptors in other cell lines (Buck et al., Biochem Pharmacol 76:1229-1239 (2008)).

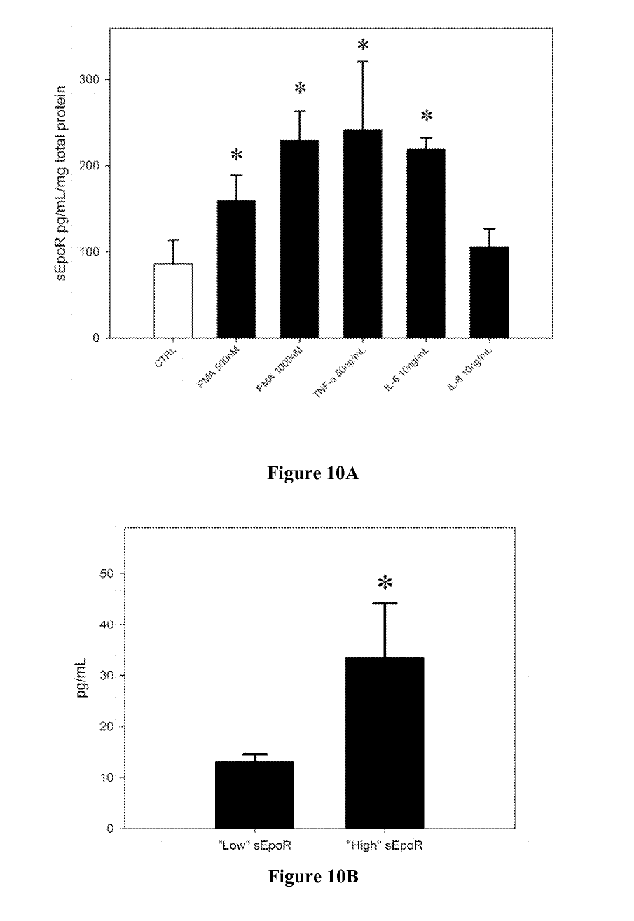

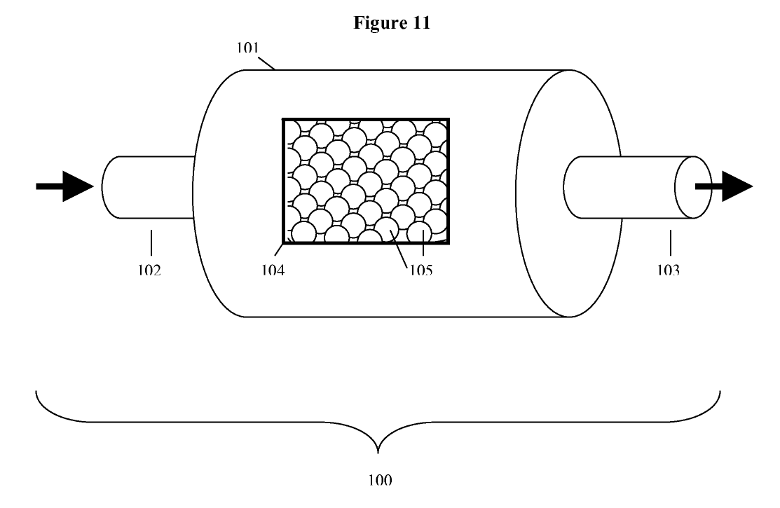

The results, shown in FIG. 10A, demonstrate that sEpoR in the cell supernatant was increased significantly over baseline by exposure to PMA, IL-6 and TNF-α but not IL-8.

Circulating levels of IL-6 were measured in a subset of patients with high sEpoR and low sEpoR; as shown in FIG. 10B, IL-6 levels were on average 2.5 times higher in subjects with high circulating levels of sEpoR than in subjects with low sEpoR levels.

These results demonstrate that serum sEpoR levels are regulated by the pro-inflammatory cytokines IL-6 and TNF-alpha.

### Example 4

### sEpoR

The sequence of a human sEpoR protein was determined as follows.

Immunoprecipitation was performed using 30 ml of pooled serum from uremic subjects using polyclonal antibody directed against EpoR (Cat #AF322, R &D Systems, MN) and immunoprecipitate samples were separated on denaturing acrylamide gels and stained with 0.1% Coomassie Brilliant Blue. A band of approximately 27-28 kDA size and a section of gel for negative control were cut out and rinsed in 1% acetonitrile. Gel fragments were subject to trypsin digest and mass spectrometry. Briefly, the samples were treated with trypsin (1:100) and resuspended in 1% trifluoroacetic acid and injected it into a CapLC (Waters) high performance liquid chromatography instrument. The peptides were separated using a 75 μm Nano Series column (LC Packings) and analyzed them using a Qstar XL MS/MS system. The peptides were searched using the Mascot search engine (Matrix Science) against the human protein database NCBInr. Using the genomic DNA from NCBI, the predicted protein sequence was determined. The predicted protein sequence shares the first 5 exons with full-length EpoR and then has 21 AA unique C-terminal piece derived from intron 5:

Human sEpoR
(SEQ ID NO: 1)
MDHLGASLWPQVGSLCLLLAGAAWAPPPNLPDPKFESKAALLAARGPEEL
LCFTERLEDLVCFWEEAASAGVGPGNYSFSYQLEDEPWKLCRLHQAPTAR
GAVRFWCSLPTADTSSFVPLELRVTAASGAPRYHRVIHINEVVLLDAPVG
LVARLADESGHVVLRWLPPPETPMTSHIRYEVDVSAGNGAGSVQRVEILE
GRTECVLSNLRGRTRYTFAVRARMAEPSFGGFWSAWSEPVSLLTPSGEAP
GGGVGGARANHGASPPP

The underlined amino acids above, i.e. GEAPGGGVGGARANHGASPPP (SEQ ID NO:2) are not present in the full length sEpoR sequence. The mRNA sequence encoding this isoform of sEpoR is as follows:

(SEQ ID NO: 3)
ATGGACCACCTCGGGGCGTCCCTCTGGCCCCAGGTCGGCTCCCTTTGTCT
CCTGCTCGCTGGGGCCGCCTGGGCGCCCCCGCCTAACCTCCCGGACCCCA
AGTTCGAGAGCAAAGCGGCCTTGCTGGCGGCCCGGGGGCCCGAAGAGCTT
CTGTGCTTCACCGAGCGGTTGGAGGACTTGGTGTGTTTCTGGGAGGAAGC
GGCGAGCGCTGGGGTGGGCCCGGGCAACTACAGCTTCTCCTACCAGCTCG
AGGATGAGCCATGGAAGCTGTGTCGCCTGCACCAGGCTCCCACGGCTCGT
GTGCGGTGCGCTTCTGGTGTTCGCTGCCTACAGCCGACACGTCGAGCTTC
GTGCCCCTAGAGTTGCGCGTCACAGCAGCCTCCGGCGCTCCGCGATATCA
CCGTGTCATCCACATCAATGAAGTAGTGCTCCTAGACGCCCCCGTGGGGC
TGGTGGCGCGGTTGGCTGACGAGAGCGGCCACGTAGTGTTGCGCTGGCTC
CCGCCGCCTGAGACACCCATGACGTCTCACATCCGCTACGAGGTGGACGT
CTCGGCCGGCAACGGCGCAGGGAGCGTACAGAGGGTGGAGATCCTGGAGG
GCCGCACCGAGTGTGTGCTGAGCAACCTGCGGGGCCGGACGCGCTACACC
TTCGCCGTCCGCGCGCGTATGGCTGAGCCGAGCTTCGGCGGCTTCTGGAG
CGCCTGGTCGGAGCCTGTGTCGCTGCTGACGCCTAGCGGTGAGGCCCCAG
GCGGGGGTGTAGGAGGAGCCAGGGCGAATCACGGGGCAAGCCCACCGCCC
TGA

An exemplary full length sequence of human EpoR is as follows:

(SEQ ID NO: 4)
MDHLGASLWPQVGSLCLLLAGAAWAPPPNLPDPKFESKAALLAARGPEEL
LCFTERLEDLVCFWEEAASAGVGPGNYSFSYQLEDEPWKLCRLHQAPTAR
GAVRFWCSLPTADTSSFVPLELRVTAASGAPRYHRVIHINEVVLLDAPVG
LVARLADESGHVVLRWLPPPETPMTSHIRYEVDVSAGNGAGSVQRVEILE
GRTECVLSNLRGRTRYTFAVRARMAEPSFGGFWSAWSEPVSLLTPSDLDP
LILTLSLILVVILVLLTVLALLSHRRALKQKIWPGIPSPESEFEGLFTTH
KGNFQLWLYQNDGCLWWSPCTPFTEDPPASLEVLSERCWGTMQAVEPGTD
DEGPLLEPVGSEHAQDTYLVLDKWLLPRNPPSEDLPGPGGSVDIVAMDEG
SEASSCSSALASKPSPEGASAASFEYTILDPSSQLLRPWTLCPELPPTPP
HLKYLYLVVSDSGISTDYSSGDSQGAQGGLSDGPYSNPYENSLIPAAEPL
PPSYVACS

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of diagnosing, or determining risk of developing, resistance to erythropoietin (EPO) in a subject, the method comprising:
selecting a subject who is being or will be treated with EPO, e.g., a subject who is undergoing or about to undergo hemodialysis, or who has anemia of chronic disease;
obtaining a sample comprising serum from the subject;
determining a level of soluble EPO receptor (sEpoR) protein in the sample;
wherein the level of sEpoR in the sample is indicative of the presence or risk of developing EPO resistance in the subject.

2. The method of claim 1, wherein the method comprises comparing the level of sEpoR in the sample to a reference.

3. The method of claim 2, wherein the presence of a level of sEpoR in the sample that is above the reference indicates that the subject has, or has an increased risk of developing, resistance to EPO.

4. The method of claim 1, wherein if the level of sEpoR indicates that the subject has, or has an increased risk of developing, resistance to EPO, the method further comprises assigning an identifier to the subject that correlates with the subject's risk.

5. The method of claim 1, wherein if the level of sEpoR indicates that the subject has, or has an increased risk of developing, resistance to EPO, the method further comprises selecting or administering an increased dose of EPO to the subject.

6. The method of claim 1, wherein if the level of sEpoR indicates that the subject has, or has an increased risk of developing, resistance to EPO, the method further comprises selecting the subject for increased monitoring.

7. The method of claim 1, wherein if the level of sEpoR indicates that the subject has, or has an increased risk of developing, resistance to EPO, the method further comprises communicating that information to the subject's health insurance provider.

8. The method of claim 1, wherein if the level of sEpoR indicates that the subject has, or has an increased risk of developing, resistance to EPO, the method further comprises administering a treatment to the subject that will lower levels of sEpoR in the serum.

9-21. (canceled)


SEQUENCE LISTING (4 sequences)

SEQ ID NO: 1 - protein, 267 aa, Homo sapiens [sequence omitted]

SEQ ID NO: 2 - protein, 21 aa, Homo sapiens
GEAPGGGVGGARANHGASPPP

SEQ ID NO: 3 - DNA, 804 bp, Homo sapiens
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcggtgaggccccaggcgggggtgtaggaggagccagggcgaatcacggggcaagcccaccgccctga

SEQ ID NO: 4 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS